(19) United States
(12) Patent Application Publication
(10) Pub. No.: US 2024/0372139 A1
(43) Pub. Date: Nov. 7, 2024

(54) DRY GEL POLYMER ELECTROLYTES

(71) Applicant: Audiance, Inc., East Greenwich, RI (US)

(72) Inventors: Michael Molinski, Tiverton, RI (US); Brian Chiou, South Kingstown, RI (US); Arijit Bose, Lexington, MA (US); Hailey Simon, Fairhaven, MA (US)

(73) Assignee: Audiance, Inc., East Greenwich, RI (US)

(21) Appl. No.: 18/682,246

(22) PCT Filed: May 2, 2022
(86) PCT No.: PCT/US2022/027275

§ 371 Date: Feb. 8, 2024

Related U.S. Application Data
(60) Provisional application No. 63/232,888, filed on Aug. 13, 2021.

(51) Int. Cl. H01M 10/0565; A61N 1/36; A61N 1/378; C08G 65/328; C08K 5/45; H01M 10/0525

(52) U.S. Cl. CPC H01M 10/0565; A61N 1/36038; A61N 1/378; C08G 65/328; C08K 5/45; H01M 10/0525; H01M 2220/30; H01M 2300/0082; H01M 2300/0085; H01M 2300/0091

(57) ABSTRACT

Dry gel polymer electrolytes as well as their methods of manufacture and use in electrochemical cells are disclosed. In some embodiments, a dry gel polymer electrolyte may include sulfolane, a high molecular weight polyethylene oxide, and a lithium salt. In embodiments in which the dry gel polymer electrolyte is included in the electrochemical cell, at least one layer of the dry gel polymer electrolyte may be disposed between an anode and a cathode of the electrochemical cell.

### CROSS-REFERENCE TO RELATED APPLICATIONS

This application claims the benefit under 35 U.S.C. § 119(e) of U.S. provisional application Ser. No. 63/232,888, filed Aug. 13, 2021, the disclosure of which is incorporated by reference in its entirety.

### FIELD

Disclosed embodiments are related to dry gel polymer electrolytes.

### BACKGROUND

Rechargeable lithium-ion batteries are commonplace in consumer electronics, electric vehicles and high-capacity energy storage systems. Lithium-ion batteries are favored due to their high gravimetric and volumetric capacity and exceptional energy densities.

### SUMMARY

In one embodiment, a dry gel polymer electrolyte includes: sulfolane; a high molecular weight polymer mixed with the sulfolane; and a lithium salt complexed with the high molecular weight polymer.

In another embodiment, an electrochemical cell includes: an anode; a cathode; and at least one dry gel polymer electrolyte layer disposed between the anode and the cathode. The at least one dry gel polymer electrolyte layer comprises a dry gel polymer electrolyte including: sulfolane; a high molecular weight polymer mixed with the sulfolane; and a lithium salt complexed with the high molecular weight polymer.

In some embodiments, an implantable medical device may include the above electrochemical cell.

In the above embodiments, the high molecular weight polymer may be a high molecular weight polyethylene oxide

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is an exploded schematic view of one embodiment of an electrochemical cell including a dry gel polymer electrolyte;

FIG. 2 is a flow diagram of one embodiment of a method for forming a dry gel polymer electrolyte;

FIG. 3 is a plot of current versus potential from linear sweep voltammetry of one embodiment of a dry gel polymer electrolyte between a voltage range of 0 to 5V at a scan rate of 1 mV/s and temperature of 37° C.;

FIG. 4 shows the lithium-ion conductivity of one embodiment of a dry gel polymer electrolyte obtained via electrochemical impedance spectroscopy;

FIG. 5 is a plot of the viscosity of one embodiment of a dry gel polymer electrolyte as a function of the applied stress between a temperature range of 5° C. to 65° C.;

FIG. 6 is a plot of the storage modulus (G′) and loss modulus (G″) of one embodiment of a dry gel polymer electrolyte versus angular frequency at a temperature of 25° C.;

FIG. 7 shows the specific discharge capacity and Coulombic efficiency of a 2016-coin cell including one embodiment of an electrochemical cell including a dry gel polymer electrolyte, a lithium titanate-based anode, and a lithium iron phosphate-based cathode over 1000 charge/discharge cycles at a charge and discharge current between 0.195 mA/cm² and 0.974 mA/cm², a voltage range of 1.0 to 2.5V, and a testing temperature of 37° C.;

FIG. 8 shows the specific discharge capacity and Coulombic efficiency of a 2 inch×3 inch single layer pouch cell including one embodiment of a dry gel polymer electrolyte, a lithium titanate-based anode, and a lithium iron phosphate-based cathode over 1800 charge/discharge cycles at a charge and discharge current between 0.215 mA/cm² and 1.07 mA/cm², a voltage range of 1.0 to 2.5V, and a temperature of 37° C.;

FIG. 9 shows the specific discharge capacity and Coulombic efficiency of a 2 inch×3 inch double layer pouch cell including one embodiment of a dry gel polymer electrolyte, two single-sided lithium titanate-based anodes, and one double-sided lithium iron phosphate-based cathode over 1400 cycles at a charge and discharge current between 0.215 mA/cm² and 1.07 mA/cm², a voltage range of 1.0 to 2.5V, and a temperature of 37° C.;

FIG. 10 shows the specific discharge capacity and Coulombic efficiency of a 0.5 inch×1.0 inch six-stack pouch cell including one embodiment of a dry gel polymer electrolyte, two single-sided and five double-sided lithium titanate-based anodes, and six double-sided lithium iron phosphate-based cathodes over 150 cycles at a charge and discharge current between 0.169 mA/cm² and 0.327 mA/cm², a voltage range of 1.0 to 2.5V, and a temperature of 37° C.;

FIG. 11 shows the specific discharge capacity and Coulombic efficiency of a 0.5 inch×1.0 inch four-stack pouch cell including one embodiment of a dry gel polymer electrolyte, two single-sided and three double-sided lithium titanate-based anodes, and four double-sided lithium iron phosphate-based cathodes over 90 cycles at a charge and discharge current 0.490 mA/cm², a voltage range of 1.0 to 2.5 V, and a temperature of 37° C.;

FIG. 12 shows the cell voltage following a nail penetration test of a 0.5 inch×1.0 inch six-stack pouch cell including one embodiment of a dry gel polymer electrolyte; and

FIG. 13 shows the cell temperature following a nail penetration test of a 0.5 inch×1.0 inch six-stack pouch cell including one embodiment of a dry gel polymer electrolyte.

### DETAILED DESCRIPTION

Traditional liquid electrolytes used in standard lithium-ion batteries are flammable, and they flow, often requiring special housings and complex battery management systems to overcome this hindrance. Solid electrolytes and carefully constructed polymer electrolytes that are non-flammable and do not flow have been used to help alleviate this drawback. However, safe, solid polymer electrolytes typically have a low lithium-ion conductivity and high interfacial resistance between the electrolyte and other components of an electrochemical cell.

In addition to the above, the Inventors have recognized a desire to use lithium-ion batteries in medical applications including, for example, cochlear implants. However, when implanting a battery in vivo, the inventors have recognized that it is desirable to avoid leakage of the electrolyte into the surrounding tissue as well as improving the thermal stability and self-extinguishing properties of the electrolyte to provide a long duration and reliable electrochemical cell for use in various in vivo applications.

In view of the foregoing, the Inventors have recognized the benefits associated with a dry gel polymer electrolyte that may exhibit self-extinguishing properties in some applications. Specifically, the Inventors have recognized the benefits associated with the electrochemical properties of sulfolane with a lithium salt as an electrolyte carrier given its desirable combination of properties including self-extinguishing characteristics, good lithium-ion transport properties, and electrically insulating properties. However, sulfolane by itself does not exhibit sufficient viscosity for the desired application as an electrolyte. Accordingly, in some embodiments, sulfolane may be combined with an appropriate polymer and lithium salt in a manner that does not significantly impact the desired properties of sulfolane. For example, in some embodiments, a high molecular weight polyethylene oxide polymer may be complexed with a lithium salt and mixed with sulfolane to provide a dry gel polymer electrolyte.

The disclosed dry gel polymer electrolytes may exhibit a desired combination of properties including, for example, improved lithium ion conductivities and self-extinguishing properties as compared to typical electrolytes. For example, in some embodiments, a dry gel polymer electrolyte as disclosed herein may have lithium-ion conductivities on the same order of magnitude as liquid electrolytes. The disclosed dry gel polymer electrolytes may also provide increased reliability and long cycle duration for electrochemical cells incorporating these materials.

In the disclosed embodiments, a high molecular weight polyethylene oxide may have any appropriate molecular weight for a desired application. For example, in some embodiments, a molecular weight of a polyethylene oxide disclosed herein may be greater than or equal to 1 Mg/mol, 2 Mg/mol, 3 Mg/mol, 5 Mg/mol, 10 Mg/mol, and/or any other appropriate molecular weight. Additionally, the molecular weight of the polyethylene oxide may be less than or equal to 15 Mg/mol, 10 Mg/mol, 5 Mg/mol, and/or any other appropriate molecular weight. Combinations of the foregoing are contemplated including, for example, a molecular weight of a polyethylene oxide polymer included in a dry gel polymer electrolyte may be between or equal to 1 Mg/mol and 15 Mg/mol. In another embodiment, the molecular weight of the polyethylene oxide polymer may be between or equal to 1 Mg/mol, and 5 Mg/mol.

Due to high molecular weight polyethylene oxide exhibiting decreased ionic conductivity relative to sulfolane, it may be desirable to limit an overall concentration of the polyethylene oxide in the dry gel polymer electrolyte. In one embodiment, a weight percentage of the polyethylene oxide in a dry gel polymer electrolyte relative to the overall weight of the dry gel polymer electrolyte may be greater than or equal to 0.5 wt %, 1 wt %, 2 wt %, 3 wt %, 4 wt %, 5 wt %, and/or any other appropriate weight percentage. Additionally, the weight percentage of the polyethylene oxide may be less than or equal to 15 wt %, 12.5 wt %, 10 wt %, 7.5 wt %, 5 wt %, 4 wt %, 3 wt %, and/or any other appropriate weight percentage. Combinations of the foregoing are contemplated including, for example, a weight percentage of polyethylene oxide and a dry gel polymer electrolyte may be between or equal to 0.5 wt % and 15 wt %. More preferably, through the use of higher molecular weights, a weight percentage of the polyethylene oxide in a dry gel polymer electrolyte may be between 1 wt % and 7.5 wt % while providing the desired viscosities and overall material properties. For example, and without wishing to be bound by theory, higher molecular weight polyethylene oxide may provide a desired viscosity at lower weight percentages as compared to lower molecular weight polyethylene oxide. In contrast, typical polyethylene oxide with a molecular weight around 100 kg/mol would not provide the viscosities disclosed herein even at weight percentages greater than 15 wt % which impact the ability of the dry gel polymer electrolyte to exhibit the desired properties.

While the current disclosure is primarily directed to dry gel polymer electrolytes including polyethylene oxide, in some embodiments, the polyethylene oxide polymer disclosed in any of the embodiments included herein may be at least partially, and in some embodiments completely, replaced with high molecular weight polyacrylonitrile (PAN), polyvinylidene fluoride (PVDF), poly (vinylidene fluoride-co-hexafluoropropylene) (PVDF-HFP), poly (methyl methacrylate) (PMMA), polydimethylsiloxane (PDMS), polyvinyl chloride (PVC), polyethylenimine (PEI), polypropylene carbonate (PPC), poly (ethylene carbonate) (PEC), poly (trimethylene carbonate) (PTMC), polypropylene glycol (PPG), polyurethane (PU), and/or combinations of the forgoing. In some embodiments, the high molecular weight polymers noted above may have molecular weights in the ranges noted above with regards to polyethylene oxide.

It should be understood that any disassociatable lithium salt able to appropriately complex with polyethylene oxide, and/or other polymers included in a dry gel polymer electrolyte, may be used with the various embodiments of a dry gel polymer electrolyte disclosed herein. For example, appropriate lithium-ion salts may include, but are not limited to, Lithium bis(trifluoromethanesulfonyl)imide (known as LiTFSI or LiN(CF₃SO₂)₂), lithium hexafluorophosphate (LiPF₆), lithium perchlorate (LiClO₄), trifluoromethanesulfonic acid lithium salt (LiCF₃SO₃), lithium tetrafluoroborate (LiBF₄), lithium bis (oxalate) borate (LiBOB), lithium bis (fluorosulfonyl) imide (LiFSI), lithium bis(pentafluoroethanesulfonyl)imide (LiBETI), lithium difluoro(oxalate)borate (LiDFOB), lithium trifluoromethansulfonate (LiTf), lithium hexfluoroarsenate (LiAsF₆), and/or any other appropriate lithium salt. In some embodiments, the lithium salt may preferably be LiTFSI. Regardless of the specific lithium salt used, the lithium salt may be present in an appropriate weight percentage relative to the overall weight of the dry gel polymer electrolyte. For example, in some embodiments, a lithium salt may be present in a weight percentage that is greater than or equal to 5 wt %, 7.5 wt %, 10 wt %, 12.5 wt %, 15 wt %, 17.5 wt %, 20 wt %, and/or any other appropriate weight percentage. Correspondingly, a weight percentage of the lithium salt in a dry gel polymer electrolyte may be less than or equal to 35 wt %, 32.5 wt %, 30 wt %, 27.5 wt %, 25 wt %, 22.5 wt %, 20 wt %, and/or any other appropriate weight percentage. Combinations of foregoing are contemplated including, for example, a weight percentage of a lithium salt in a dry gel polymer electrolyte that is between or equal to 5 wt % and 35 wt %. More preferably, a weight percentage of the lithium salt in a dry gel polymer electrolyte may be between 15 wt % and 25 wt % while providing the desired viscosities and overall material and electrochemical properties.

As elaborated on further below, the disclosed dry gel polymer electrolytes may be manufactured using a high shear mixing process. Accordingly, to help facilitate the shear mixing of a mixture, in some embodiments, a dry gel polymer electrolyte may include an appropriate shear media dispersed therein. Appropriate types of shear media may include, but are not limited to, zirconia shear media, alumina shear media, other ceramic shear media, combinations of the forgoing, tungsten carbide shear media, stainless steel shear media, agate shear media, silicon carbide shear media, silicon nitride shear media and/or any other appropriate shear media that is electrochemically stable within the dry gel polymer electrolyte during operation. In some embodiments, the shear media may correspond to particles of a desired material dispersed in the electrolyte mixture. The shear media particles may have shapes including, but not limited to, spheres, oblong shapes, cylindrical, combinations of the foregoing, and/or any other appropriate shape as the disclosure is not so limited. Appropriate average particle sizes (e.g. an average maximum transverse dimension of the particles) may be between or equal to 0.1 mm and 40 mm for spherical shear media, and 5.5 mm and 15 mm for cylindrical shear media. Additionally, a weight percentage of the shear media relative to the weight of an overall dry gel polymer electrolyte may be greater than or equal to 0 wt %, 2 wt %, 4 wt %, 6 wt %, 8 wt %, 10 wt %, and/or any other appropriate weight percentage. The weight percentage of the shear media in the dry gel polymer electrolyte may also be less than or equal to 25 wt %, 24 wt %, 22 wt %, 20 wt %, 18 wt %, 16 wt %, 14 wt %, 12 wt %, and/or any other appropriate weight percentage. Combinations of the foregoing are contemplated including, for example, a weight percentage of a shear media in a dry gel polymer electrolyte may be between or equal to 0 wt % and 25 wt %. In another embodiment, a weight percentage of the shear media may be between or equal to 2 wt % and 25 wt %.

In some embodiments, a weight percentage of sulfolane in a dry gel polymer electrolyte relative to an overall weight of the dry gel polymer electrolyte may be greater than or equal to 55 wt %, 60 wt %, 65 wt %, 70 wt %, and/or any other appropriate weight percentage. Correspondingly, a weight percentage of the sulfolane in the dry gel polymer electrolyte may be less than or equal to 85 wt %, 80 wt %, 75 wt %, 70 wt %, and/or any other appropriate weight percentage. Combinations of the foregoing are contemplated including. for example, a weight percentage of sulfolane in the dry gel polymer electrolyte may be between or equal to 55 wt % and 85 wt %. More preferably, a weight percentage of the lithium salt in a dry gel polymer electrolyte may be between 65 wt % and 80 wt % while providing the desired viscosities and overall material and electrochemical properties.

The various embodiments of dry gel polymer electrolytes disclosed herein may exhibit any desired range of viscosities for a given application. That said, in some embodiments, a viscosity of a dry gel polymer electrolyte as disclosed herein may be greater than or equal to 10 Pa·s, 100 Pa·s, 200 Pa·s, 300 Pa·s, 400 Pa·s, 500 Pa·s, and/or any other appropriate viscosity at a measurement temperature of 25° C. The viscosity of the dry gel polymer electrolyte may also be less than or equal to 1000 Pa·s, 900 Pa·s, 800 Pa·s, 700 Pa·s, 600 Pa·s, 500 Pa·s, and/or any other appropriate viscosity at the measurement temperature. Combinations of the foregoing are contemplated including, for example, a viscosity may be between or equal to 10 Pa·s and 1000 Pa·s at a measurement temperature of 25° C. In some embodiments, the disclosed dry gel polymer electrolytes may be shear thinning materials. The viscosities at the noted measurement temperatures may be measured using a stress-controlled TA Instruments Discovery hybrid rheometer (HR20) with a Peltier temperature controller or other similar system.

The various embodiments of dry gel polymer electrolytes may exhibit any desired range of lithium-ion conductivities for a given application. That said, in some embodiments, a lithium-ion conductivity of a dry gel polymer electrolyte as disclosed herein may be greater than or equal to 10⁻⁵ S/cm, 10⁻⁴ S/cm, 10⁻³ S/cm and/or any other appropriate lithium-ion conductivity at a measurement temperature of 25° C. The lithium-ion conductivity of the dry gel polymer electrolyte may also be less than or equal to 10⁻² S/cm, 10⁻³ S/cm, 10⁻⁴ S/cm, and/or any other appropriate lithium-ion conductivity at the measurement temperature. Combinations of the foregoing are contemplated including, for example, a lithium-ion conductivity of a dry gel polymer electrolyte as disclosed herein may be between or equal to 10⁻⁵ S/cm and 10⁻² S/cm at a measurement temperature of 25° C. The lithium-ion conductivities at the noted measurement temperatures may be measured using electrochemical impedance spectroscopy at 25° C.

As noted above, in some embodiments, a dry gel polymer electrolyte may exhibit a desired combination of flammability characteristics for a particular application. For example, in some embodiments, a dry gel polymer electrolyte as disclosed herein may be self-extinguishing under normal atmospheric conditions at sea level. In other words, if the electrolyte were exposed to an ignition source resulting in burning of the dry gel polymer electrolyte, once the ignition source is removed, the dry gel polymer electrolyte would not continue to burn and would self-extinguish after the ignition source is removed when exposed to normal atmospheric conditions at standard temperature and pressure.

While specific weight percentages of the different components and properties of the resulting dry gel polymer electrolytes are detailed above, it should be understood that the current disclosure is not limited to only these ranges and that weight percentages and material properties both greater than and less than the ranges noted above are also contemplated as the disclosure is not limited in this fashion.

As elaborated on in regards to the figures, in some embodiments, the dry gel polymer electrolytes disclosed herein may be incorporated into an electrochemical cell, such as a lithium-ion battery, and in some instances an all-solid lithium-ion battery. In some embodiments, at least one layer of a dry gel polymer electrolyte may be disposed between an anode and cathode of the electrochemical cell. The dry gel polymer electrolyte layer may be applied either as a separately formed layer, or the dry gel polymer electrolyte may be applied onto the anode and/or cathode. For example, doctor blading, extrusion, spreading, and/or any other appropriate way of applying and/or otherwise forming a layer of dry gel polymer electrolyte in a semi-liquid state may be used as the disclosure is not limited to how the desired layers may be formed or applied to the surface of an active layer of an electrochemical cell.

Depending on the embodiment, a layer of dry gel polymer electrolyte may have any appropriate thickness for a desired application. For instance, in some embodiments, a layer of dry gel polymer electrolyte may have a thickness that is greater than or equal to 5 μm, 15 μm, 20 μm, 25 μm, and/or any other appropriate thickness. Correspondingly, the thickness may be less than or equal to 100 μm, 75 μm, 50 μm, and/or any other appropriate thickness. Combinations of the foregoing are contemplated including, for example, a thickness that is between or equal to 5 μm and 100 μm. Of course, thicknesses both greater than and less than those noted above are also contemplated as the disclosure is not so limited.

The inventors have recognized that for implantable applications, as well as other potential applications, it may be desirable to limit the expansion and contraction of the anode and/or cathode of an electrochemical cell to limit particle degradation and improve cycle life for an electrochemical cell. For example, nickel manganese cobalt and graphite based active materials exhibit relatively large volume changes during lithiation and delithiation. Accordingly, in some embodiments, it may be desirable to use anode and/or cathode materials that exhibit decreased swelling during cycling to further improve the reliability of the resulting electrochemical cell. For example, in some embodiments, it may be desirable to provide less than 5% swelling between complete lithiation and delithiation of an electrochemical cell. In one such embodiment, an electrochemical cell may include an anode including a lithium titanate material and a cathode including a lithium iron phosphate material.

While a specific beneficial electroactive material combination is provided above, it should be understood that any appropriate combination of electroactive materials may be used in an electrochemical cell. For example, in some embodiments, an anode material may be selected from lithium titanate (Li₄Ti₅O₁₂; LTO), silicon (Si), graphite (C), lithium (Li), tin (Sn), germanium (Ge), combinations of the foregoing, and/or any other appropriate anode active material. Additionally, a cathode active material may include lithium iron phosphate (LiFePO₄, LFP), lithium cobalt oxide (LiCoO₂; LCO), lithium nickel manganese cobalt oxide (LiNiₓMn_{y}Co_{1-x-y}O₂; NMC333, NMC532, NMC622, or NMC811), lithium nickel cobalt aluminum oxide (LiNi_{0.8}Co_{0.15}Al_{0.05}O₂; NCA), lithium manganese oxide (LiMn₂O₄; LMO), lithium nickel manganese oxide (LiNi_{0.5}Mn_{1.5}O₄; LNMO), combinations of the foregoing. and/or any other appropriate cathode active material.

It should be understood that an electrochemical cell as disclosed herein may include any appropriate additives binders, or other appropriate materials as part of the anode and/or cathode constructions of the electrochemical cell. For example, the anode and cathode materials may be deposited onto an appropriate current collector made from a metal foil such as an aluminum foil, copper foil, or other appropriate conductive substrate. Conductive additives such as conductive carbon, carbon coated active material particles, carbon nanotubes, graphene, and other conductive additives may be included in an anode and/or cathode. Similarly, a binder may be included in the anode and/or cathode. Appropriate binders may include, but are not limited to a conductive polymer with a relatively low melting point such as poly (vinylidene difluoride) (PVDF), poly (vinylidene difluoride-hexafluoropropylene) PVDF-HFP, poly (3,4-ethylenedioxythiophene) (PEDOT:PSS), polyacrylic acid (PAA), polytetrafluoroethylene (PTFE), sodium carboxymethyl cellulose/styrene butadiene rubber (CMC/SBR), polyvinyl alcohol (PVA), polyaniline (PANi), polyethylenimine (PEI), polyethylene oxide (PEO), poly (methyl methacrylate) (PMMA), polyacrylonitrile (PAN), polyimide (PI), polyurethane (PU), sodium alginate (SA), polyacetylene (PA), poly (methyl acrylate) (PMA), polystyrene (PS) and/or any other appropriate binder.

The anode and cathode of an electrochemical cell may be prepared and applied to a corresponding current collector in any appropriate manner. For example, dry mixing of the electrode powders with appropriate solvents, binders, and additives may be used in some embodiments. The resulting mixture may then be deposited onto the corresponding current collector using slurry casting techniques such as a standard doctor blading process or any other standard deposition technique used to form electrochemical cell electrodes. The N/P ratio may ideally be between 1.0 to 1.5, and more preferably between 1.1 and 1.3. The resulting electrochemical cell electrodes may be stacked into a desired multi-stack parallel or series configuration to meet application specific capacity or voltage requirements.

Electrochemical cells disclosed herein may exhibit any appropriate size and/or shape factor. For example, in some embodiments, an electrochemical cell may have a coin cell construction, a prismatic construction, a pouch cell construction, a stack plate construction, a jellyroll construction, and/or any other appropriate cell construction and/or size as the disclosure is not so limited. For example, the positive and negative electrodes of an electrochemical cell may be stacked, wound, or rolled in a parallel or series multi-stack configuration. Additionally, while the disclosed dry gel polymer electrolytes may be especially beneficial in all solid electrochemical cells, embodiments in which the dry gel polymer electrolytes are used in other cell constructions, or any other appropriate use, are also contemplated. The electrochemical cells made with the dry gel polymer electrolytes disclosed herein may also be used for any desired application including, for example, automotive batteries, batteries for consumer electronics, medical devices, and/or any other application. With regards to medical devices, the disclosed electrochemical cells may be configured to be implanted as part of an implantable medical device that may be implanted in vivo within a subject.

Electrochemical cells disclosed herein may exhibit any desired combination of performance characteristics based on the specific sizing of the electrochemical cell and electroactive materials incorporated into the electrochemical cell. That said, the disclosed electrochemical cells may be capable of operating at charging rates between C/10 and 1 C for at least 500, 750, 1000, 1200, 1800, and/or any other appropriate number of charge/discharge cycles while exhibiting a capacity drop of less than or equal to 80% relative to an initial capacity of the electrochemical cell. Additionally, the disclosed electrochemical cells may exhibit a temperature rise relative to a surrounding ambient temperature of less than or equal to 2 Celsius at a charge/discharge rate of C/10. The electroactive materials of the electrochemical cells disclosed herein may also be selected to permit long duration operation in environments corresponding to the internal body temperature of a subject (e.g., 37° C.). Of course, while specific operating parameters and benefits are noted above, in other embodiments, electrochemical cells exhibiting different operating parameters and/or benefits are also contemplated as the disclosure is not limited in this fashion.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described hercin.

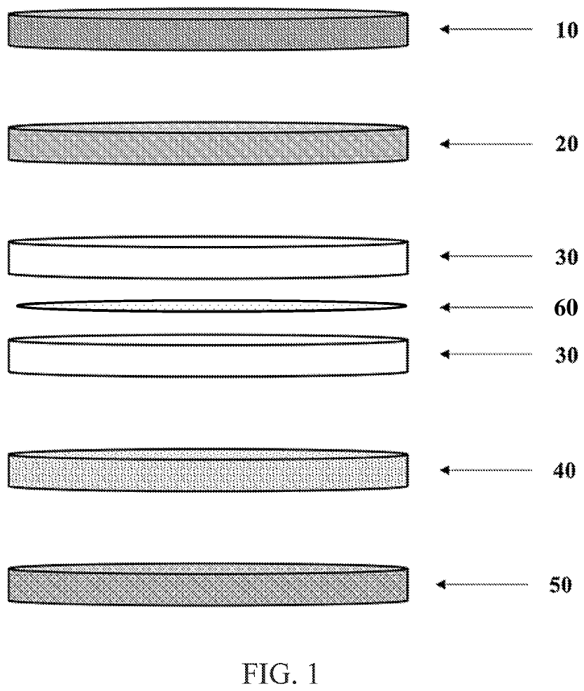

FIG. 1 illustrates an exploded schematic diagram of one embodiment of an electrochemical cell including the dry gel polymer electrolytes disclosed herein. In the depicted embodiment, the electrochemical cell includes an anode current collector 10, an anode material 20 disposed on the anode current collector 10, a cathode current collector 50, and a cathode material 40 disposed on the cathode current collector 50. At least one layer of a dry gel polymer electrolyte 30 may be disposed between the anode and cathode of the electrochemical cell. In some embodiments, a single layer of dry gel polymer electrolyte is disposed directly between the anode and cathode. However, in other embodiments, a first layer of the dry gel polymer electrolyte 30 may be applied to a surface of the anode 20 and a second layer of the dry gel polymer electrolyte may be applied to an opposing surface of the cathode 40 with a separator 60 disposed between the two layers of the dry gel polymer electrolyte. The separator may correspond to a typical mechanical separator used in electrochemical cells. It should be understood that while coin cell geometries for the various components of the electrochemical cell are illustrated in the figure, other geometries for the electrochemical cell may also be used as the disclosure is not so limited.

The cathode material 40 and anode material 20 may be deposited on the corresponding current collector 50 and 10 respectively (which may be a metal foil such as an aluminum and/or copper foil). These materials may be applied onto the current collector's using any appropriate application method including, for example, doctor-blading a slurry onto the current collectors to provide a given thickness of material and placing the anode and cathode on a vacuum bed to remove the solvent. The coating thicknesses may be adjusted to obtain electrode balancing and desired mass loadings. The drying of the cathode and anode films may be completed at a temperature which, preferably, is below the melting temperature of the polymer comprising the dry gel polymer electrolyte. A steel rule die or disc cutter of desired diameter may be used to cut the cathode and anode films into the appropriate size and shape, including the depicted circular geometries shown in the figure, though other geometries, including uncut foils for winding, may also be used. The cathode and anode sheets or discs may be densified using isostatic compression prior to assembly of the different layers in some embodiments.

In some embodiments, the densification of the anode and cathode layers may be conducted at a temperature below a melting temperature of a dry gel polymer electrolyte. Subsequently, a temperature of the dry gel polymer electrolyte may be increased to a temperature that is greater than a melting temperature of the dry gel polymer electrolyte during pressing of the dry gel polymer electrolyte between the anode and cathode materials. However, embodiments in which a pressing process is conducted below a melting temperature of the dry gel polymer electrolyte are also contemplated. Appropriate pressures for densifying the various layers and/or for pressing the layers together may be between or equal to 0.01 MPa and 200 MPa, or more preferably between 10 MPa and 50 MPa, though other pressures may also be used.

It should be understood that the above-noted processing steps may be conducted using pre-mixed powder compositions for the anode and cathode materials and the various processes may be conducted in a low humidity environment. For example, the disclosed processes may be conducted in an environment having a relative humidity of less than 2% in some embodiments. Also, while slurry-based casting methods are disclosed above with regards to the anode and cathode, the current disclosure is not limited to how the various active material layers are formed or assembled together.

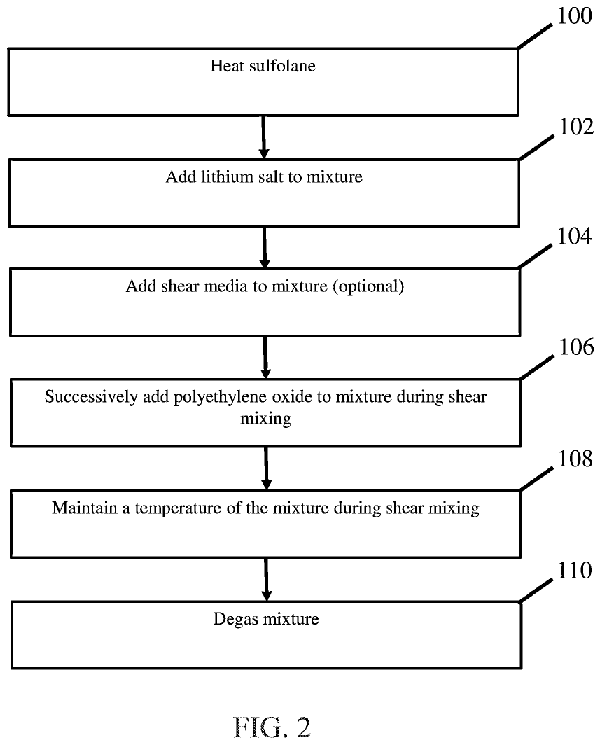

FIG. 2 depicts one embodiment of a method for manufacturing a dry gel polymer electrolyte. In the depicted embodiment, sulfolane is heated at 100. In some embodiments, a temperature of the sulfolane may be between or equal to 40° C. and 60° C. At 102, one or more lithium salts are added to the sulfolane. At 104 shear media, or any other appropriate additive, may optionally be added to the mixture to facilitate shear mixing of the dry gel polymer electrolyte. The high molecular weight polyethylene oxide may be successively added in small batches to the heated sulfolane during high shear mixing. In some embodiments, the high shear mixing may be conducted for a time period between or equal to 30 minutes and 240 minutes though any appropriate time period for providing a uniform composition for the dry gel polymer electrolyte for a giving type of mixing may also be used. Appropriate devices for applying the desired high shear mixing may include, but are not limited to, 1-3 shaft mixer with a high shear blade, planetary mixer, centrifugal mixer, and planetary centrifugal mixer. Additionally, the shear rates applied to the material during mixing may be between or equal to 10 1/s and 100000 1/s, though other shear rates both greater and less than those noted above may also be used. At 108, a temperature of the mixture may be maintained within a desired temperature range during mixing. In some embodiments, the shear applied to the mixture may be appropriately balanced with heat losses from the system to both initially head and maintain the mixture in the desired temperature range of 40° C. to 60° C. in steps 100 and 108. However, embodiments in which an active heater and/or cooler is used to actively control a temperature of the mixture are also contemplated. After adding all of the desired materials and mixing them to provide a uniform composition, the solution may be degassed at 110 by exposing the dry electrolyte polymer to a reduced pressure while at an appropriate elevated temperature with a sufficiently low viscosity to permit trapped gases to be drawn out of the molten electrolyte. In some embodiments, this thermal treatment and degassing of the dry gel polymer electrolyte may be conducted at a temperature that is greater than a melting temperature of the dry gel polymer electrolyte and a pressure that is less than an ambient pressure. Similar to other electrochemical manufacturing processes, the various steps of the above-described method may be performed in a dry environment, which may correspond to an environment comprising less than 2% relative humidity in some embodiments.

### Example: Electrolyte Composition and Electrochemical Cell Construction

In one exemplary embodiment, an anode current collector, may be comprised of copper. The anode may be comprised of an active material (Li₄Ti₅O₁₂), conductive carbon (Super C45, Super P. Carbon Nanotubes, and/or KS6), a binder (PVDF) and/or a polymer (PEO) complexed with a lithium salt (LiN(CF₃SO₂)₂) and carrier sulfolane. The dry gel polymer electrolyte separating the composite anode and cathode may be comprised of polyethylene oxide (PEO) with a lithium salt (LiN(CF₃SO₂)₂) and carrier sulfolane. The cathode may be comprised of an active material (LiFePO₄), conductive carbon (Super C45, Super P. Carbon Nanotubes, and/or KS6), a binder (PVDF) and/or an ionically conductive polymer (PEO) complexed with a lithium salt (LiN (CF₃SO₂)₂) and carrier sulfolane. The cathode current collector 50 may be comprised of aluminum.

### Example: Electrolyte Composition and Electrode Compositions

In another exemplary embodiment, a dry gel polymer electrolyte of an electrochemical cell may include polymer/salt complexes with high molecular weight PEO as the ionically conductive polymer and LiN(CF₃SO₂)₂, mixed with sulfolane where sulfolane accounts for 76 wt % of the dry gel polymer electrolyte and the ratio of salt to sulfolane is 8:2 by weight. The anode may comprise 84.00 wt % Li₄Ti₅O₁₂, 12.0% Super P, and 4.00% PVDF (all by weight). The cathode may comprise 84.00% LiFePO₄, 12.0% Super P, and 4.00% PVDF (all by weight).

### Example: Electrolyte Composition and Electrode Compositions

In another exemplary embodiment, a dry gel polymer electrolyte of an electrochemical cell may include polymer/salt complexes with high molecular weight PEO as the ionically conductive polymer and LiN(CF₃SO₂)₂, mixed with sulfolane where sulfolane accounts for 76 wt % of the dry gel polymer electrolyte and the ratio of salt to sulfolane is 8:2 by weight. The anode may comprise 90.00 wt % Li₄Ti₅O₁₂, 5.2% Super P, 0.8% carbon nanotubes, and 4.00% PVDF (all by weight). The cathode may comprise 90.00% LiFePO₄, 5.2% Super P, 0.8% carbon nanotubes, and 4.00% PVDF (all by weight).

### Example: Electrolyte Composition and Electrode Compositions

In another exemplary embodiment, a dry gel polymer electrolyte of an electrochemical cell may include polymer/salt complexes with high molecular weight PEO as the ionically conductive polymer and LiN(CF₃SO₂)₂, mixed with sulfolane where sulfolane accounts for 71 wt % of the dry gel polymer electrolyte and the ratio of salt to sulfolane is 75:25 by weight. The anode may comprise 90.00 wt % Li₄Ti₅O₁₂, 5.2% Super P, 0.8% carbon nanotubes, and 4.00% PVDF (all by weight). The cathode may comprise 90.00% LiFePO₄, 5.2% Super P, 0.8% carbon nanotubes, and 4.00% PVDF (all by weight).

### Example: Electrolyte Composition and Electrode Compositions

In another exemplary embodiment, a dry gel polymer electrolyte of an electrochemical cell may include polymer/salt complexes with high molecular weight PEO as the ionically conductive polymer and LiN(CF₃SO₂)₂, mixed with sulfolane where sulfolane accounts for 71 wt % of the dry gel polymer electrolyte and the ratio of salt to sulfolane is 75:25 by weight. The anode may comprise 90.00 wt % Li₄Ti₅O₁₂, 1.0% Super P, 2.1% Super C45, 2.1% KS6, 0.8% carbon nanotubes, and 4.00% PVDF (all by weight). The cathode may comprise 90.00% LiFePO₄, 1.0% Super P, 2.1% Super C45, 2.1% KS6, 0.8% carbon nanotubes, and 4.00% PVDF (all by weight).

### Example: Stability of PEO

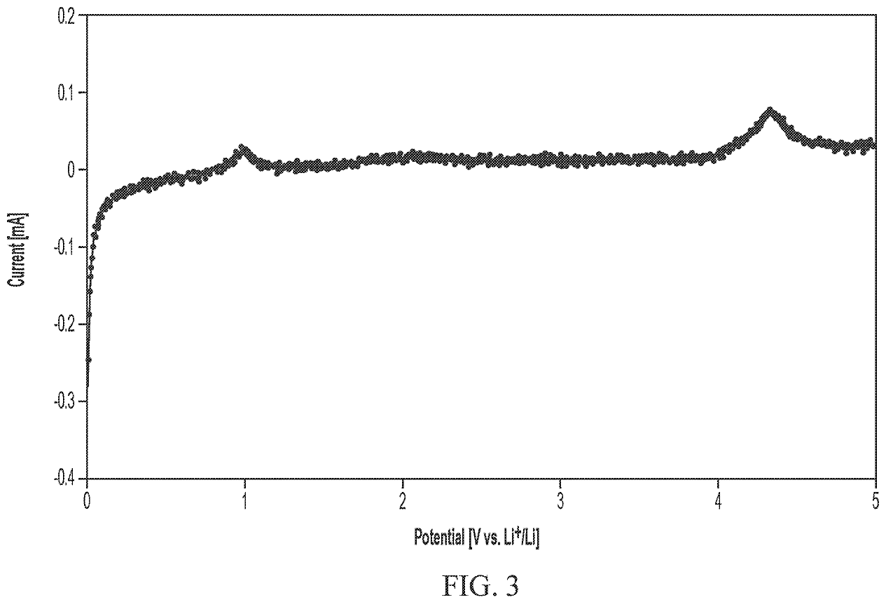

FIG. 3 is a plot of current versus potential from linear sweep voltammetry of one embodiment of a dry gel polymer electrolyte including polyethylene oxide (PEO) between a voltage range of 0 to 5V at a scan rate of 1 mV/s and temperature of 37° C. The testing arrangement included a metallic lithium reference against a stainless steel counter electrode. As shown in the figure, the linear sweep voltammetry shows a wide electrochemical stability of the dry gel polymer electrolyte including PEO up to 5V. Without wishing to be bound by theory, the peaks at approximately 1V and 4.25V represent dissolution of lithium into the PEO and oxidation of PEO respectively.

### Example: Ionic Conductivity

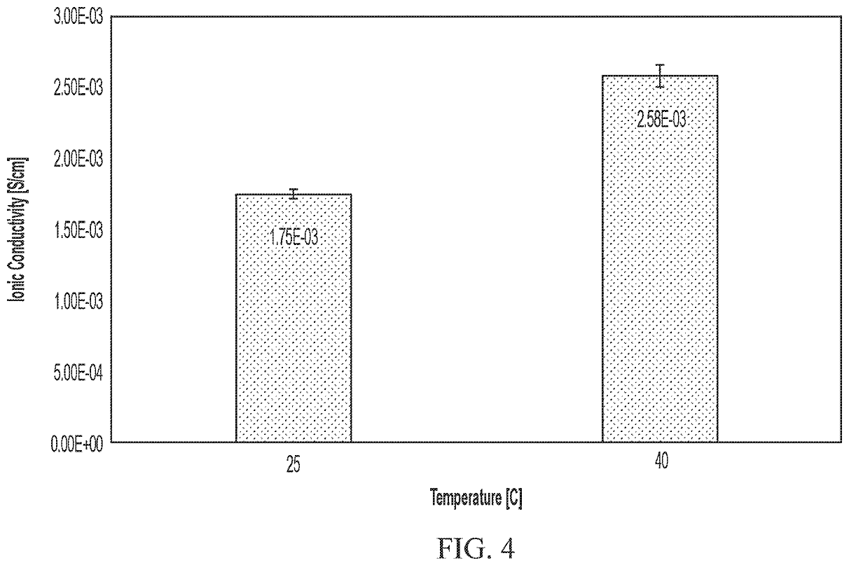

FIG. 4 shows the lithium-ion conductivity of one embodiment of a dry gel polymer electrolyte including polyethylene oxide (PEO) obtained via electrochemical impedance spectroscopy. Specifically, the ionic conductivity of a dry gel polymer electrolyte including PEO, LIN(CF₃SO₂)₂ as the lithium salt, and sulfolane was measured. The ratio of lithium salt to sulfolane was maintained at 8:2 and the weight percentage of sulfolane was 76% of the total dry gel polymer electrolyte. The ionic conductivity was measured at 20° C. and 40° C. The ionic conductivities were determined using Electrochemical Impedance Spectroscopy (EIS) on a Princeton Applied Research VersaSTAT 3 Potentiostat Galvanostat. The test was conducted using a frequency range of 1 Mhz to 0.01 hz with an excitation amplitude of 10.0 mV. The dry gel polymer electrolyte exhibited a temperature dependence for the ionic conductivity of about 1.75×10⁻³ S/cm at 25° C. and 2.58×10⁻³ S/cm at 40° C.

### Example: Viscosity

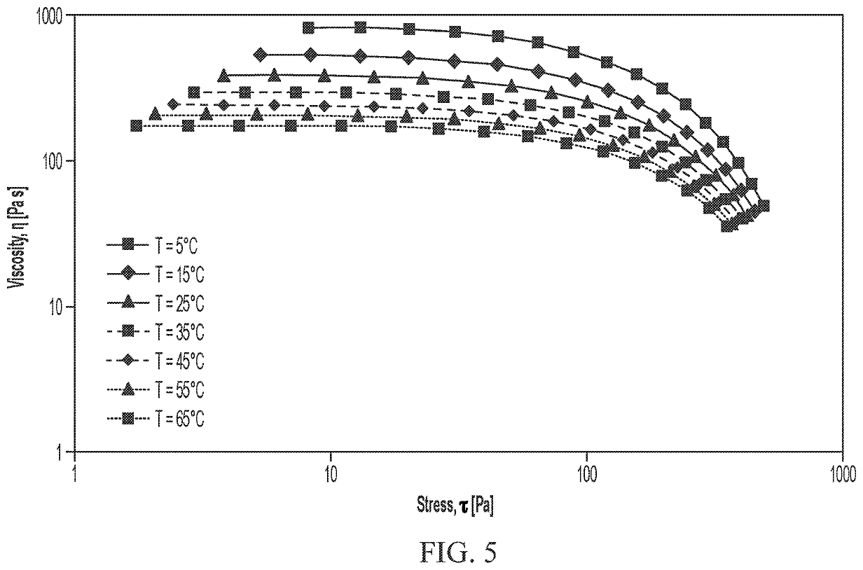

The viscosity versus shear stress (τ) of a dry gel polymer electrolyte at temperatures between 5° C. and 65° C. is shown in FIG. 5. The composition was the same as described above regarding the ionic conductivity measurements and the viscosity measurements were taken using a stress-controlled TA Instruments Discovery hybrid rheometer (HR20) with a Peltier temperature controller. The dry gel polymer electrolyte was observed to exhibit shear-thinning behavior. The dry gel polymer electrolyte exhibited viscosities ranging between about 100 Pa·s and 1000 Pa·s in the zero-strain rate limit for tested temperatures.

### Example: Storage and Loss Modulus

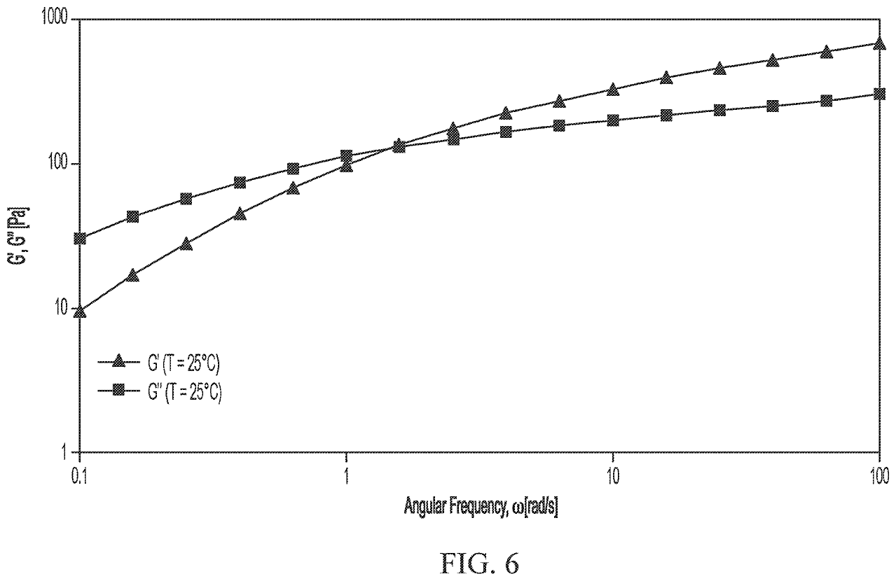

As shown in FIG. 6, the measured storage (G′), loss modulus (G″), and complex viscosity was plotted against the angular frequency at a testing temperature of 25° C. Without wishing to be bound by theory, the dominance of the loss modulus at low angular frequencies and storage modulus at high angular frequencies represents a transition from liquid-like to solid-like behavior at approximately 1 rad/sec giving a relaxation time constant of approximately 3 sec for this electrolyte. This behavior is characteristic of polymer electrolytes. The above testing was conducted using materials with compositions similar to those described above for the ionic conductivity measurements and the measurements were taken using a stress-controlled TA Instruments Discovery hybrid rheometer (HR20) with a Peltier temperature controller.

### Example: Electrochemical Cell Capacity and Efficiency

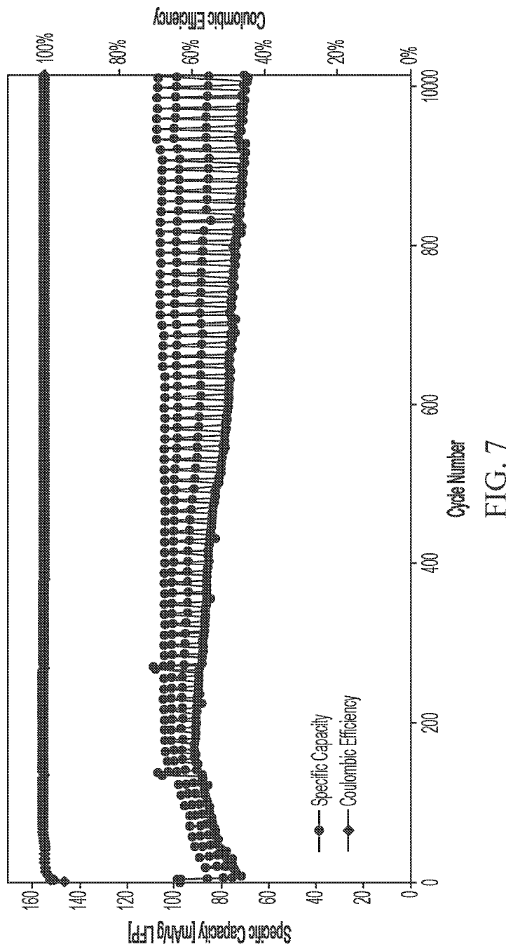

FIG. 7 shows the discharge capacity and Coulombic efficiency versus number of cycles for a 2016-coin cell. The dry gel polymer electrolyte included polymer/salt complexes with PEO as the polymer, LiN(CF₃SO₂)₂ as the lithium salt, and sulfolane as the carrier. The anode comprised of Li₄Ti₅O₁₂ as an active material, Super P and carbon nanotubes as a conductive carbon, and PVDF as a binder. The cathode comprised Li₄FePO₄ as an active material, Super P and carbon nanotubes as a conductive carbon, and PVDF as a binder. Charge/discharge cycling was completed at 37° C. The battery underwent over 1000 charge/discharge cycles at a charge and discharge current between 0.195 and 0.974 mA/cm₂ and a voltage range between 1.0 and 2.5 V. The cycling profile followed a conditioning step of 3 cycles at 0.10 C, followed by a continuous loop of 0.20 C (1 cycle), 0.30 C (1 cycle), 0.40 C (1 cycle), and 0.50 C (10 cycles) with all charging steps at CCCV (constant current-constant voltage).

### Example: Electrochemical Cell Capacity and Efficiency

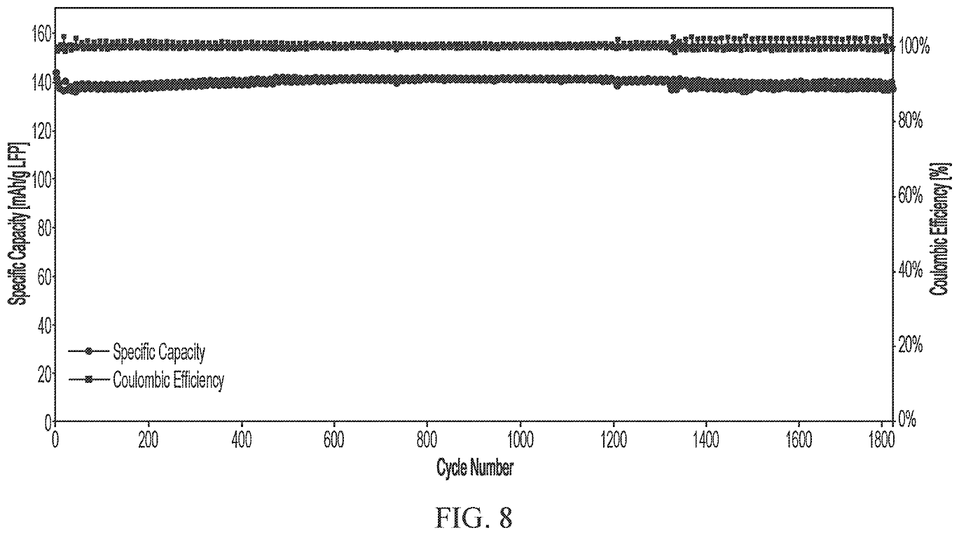

FIG. 8 shows the discharge capacity and Coulombic efficiency from a 2×3 inch pouch cell. The polymer/salt complexes have PEO as the ionically conductive polymer, LiN(CF₃SO₂)₂ as the lithium salt, and sulfolane as the carrier. The anode comprised Li₄Ti₅O₁₂ as an active material, Super P and carbon nanotubes as a conductive carbon, and PVDF as a binder. The cathode comprised Li₄FePO₄ as an active material, Super P and carbon nanotubes as a conductive carbon, and PVDF as a binder. Charge/discharge cycling was completed at 37° C. for over 1800 cycles at a charge and discharge current between 0.215 and 1.07 mA/cm² over a voltage range of 1.0 to 2.5V. The cycling profile followed a conditioning step of 3 cycles at 0.10 C, followed by a continuous loop of 0.20 C (1 cycle), 0.30 C (1 cycle), 0.40 C (1 cycle), and 0.50 C (10 cycles) with all charging steps at CCCV (constant current constant voltage).

### Example: Electrochemical Cell Capacity and Efficiency

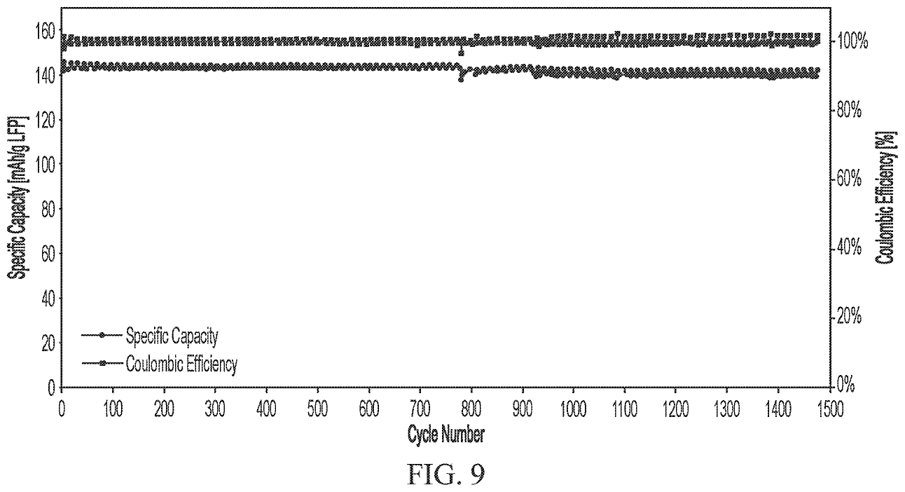

FIG. 9 shows the discharge capacity and Coulombic efficiency from a 2×3 inch double layer pouch cell including one embodiment of a dry gel polymer electrolyte, two single-sided lithium titanate-based anodes, and one double-sided lithium iron phosphate-based cathode. The polymer/salt complexes have PEO as the ionically conductive polymer, LiN(CF₃SO₂)₂ as the lithium salt, and sulfolane as the carrier. The anode comprised Li₄Ti₅O₁₂ as an active material, Super P and carbon nanotubes as a conductive carbon, and PVDF as a binder. The cathode comprised Li₄FePO₄ as an active material, Super P and carbon nanotubes as a conductive carbon, and PVDF as a binder. Charge/discharge cycling was completed at 37° C. for over 1400 cycles at a charge and discharge current between 0.215 and 1.07 mA/cm² over a voltage range of 1.0 to 2.5V. The cycling profile followed a conditioning step of 3 cycles at 0.10 C, followed by a continuous loop of 0.20 C (1 cycle), 0.30 C (1 cycle), 0.40 C (1 cycle), and 0.50 C (10 cycles) with all charging steps at CCCV (constant current constant voltage).

### Example: Electrochemical Cell Capacity and Efficiency

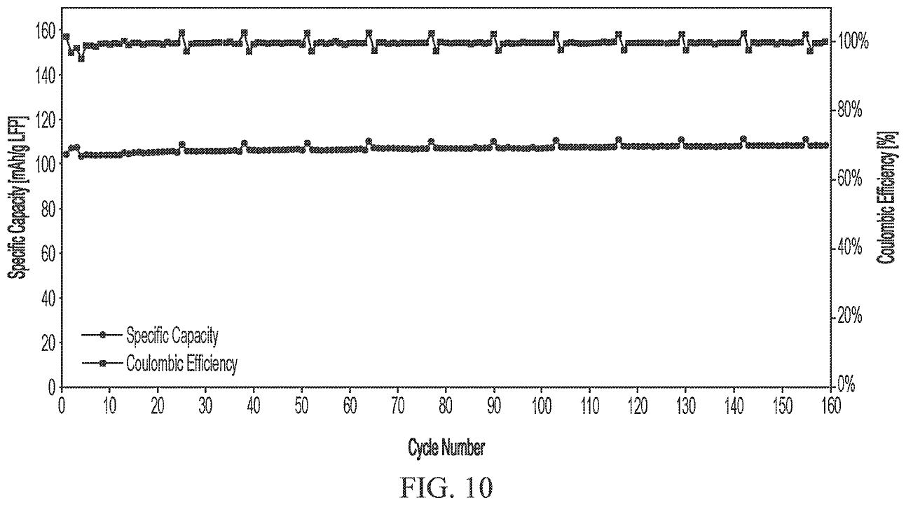

FIG. 10 shows the specific discharge capacity and Coulombic efficiency of a 0.5″×1.0″ six-stack pouch cell including one embodiment of a dry gel polymer electrolyte, two single-sided and five double-sided lithium titanate-based anodes, and six double-sided lithium iron phosphate-based cathodes. The polymer/salt complexes have PEO as the ionically conductive polymer, LiN(CF₃SO₂)₂ as the lithium salt, and sulfolane as the carrier. The anode comprised Li₄Ti₅O₁₂ as an active material, Super P and carbon nanotubes as a conductive carbon, and PVDF as a binder. The cathode comprised Li₄FePO₄ as an active material, Super P and carbon nanotubes as a conductive carbon, and PVDF as a binder. The active material and Super P were dry mixed prior to carbon nanotube and PVDF addition for both anode and cathode. Charge/discharge cycling was completed at 37° C. for over 150 cycles at a charge and discharge current between 0.169 mA/cm² and 0.327 mA/cm² over a voltage range of 1.0 to 2.5V. The cycling profile followed a conditioning step of 3 cycles at 0.10 C, followed by 0.10 C (3 cycles), 0.19 C (9 cycles), and then a continuous loop of 0.19 C (10 cycles), and 0.10 C (1 cycle), with all charging steps at CCCV (constant current constant voltage).

### Example: Electrochemical Cell Capacity and Efficiency

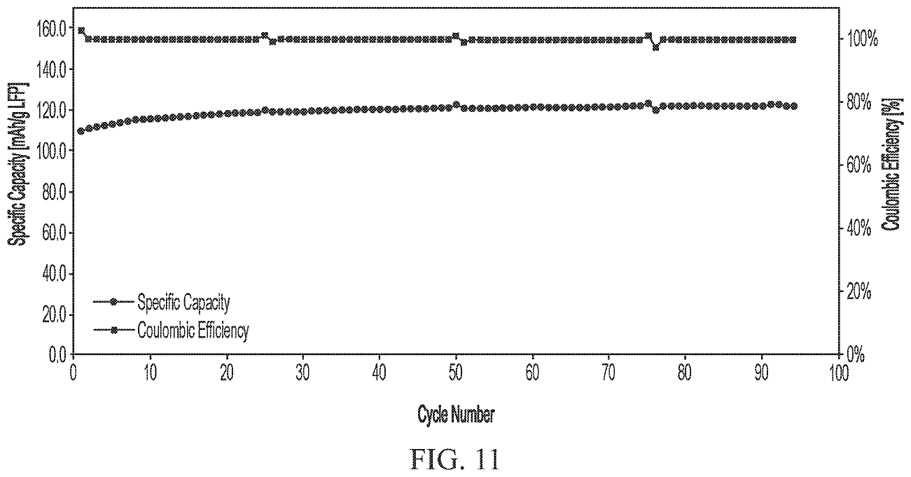

FIG. 11 shows the specific discharge capacity and Coulombic efficiency of a 0.5″×1.0″ four-stack pouch cell including one embodiment of a dry gel polymer electrolyte. two single-sided and three double-sided lithium titanate-based anodes, and four double-sided lithium iron phosphate-based cathodes. The polymer/salt complexes have PEO as the ionically conductive polymer, LiN(CF₃SO₂)₂ as the lithium salt, and sulfolane as the carrier. The anode comprised Li₄Ti₅O₁₂ as an active material, Super P, Super C45, KS6, and carbon nanotubes as conductive carbons, and PVDF as a binder. The cathode comprised Li₄FePO₄ as an active material, Super P, Super C45, KS6, and carbon nanotubes as conductive carbons, and PVDF as a binder. The active material, Super P, and Super C45 were dry mixed prior to KS6, carbon nanotube, and PVDF addition for both anode and cathode. Charge/discharge cycling was completed at 37° C. for over 90 cycles at a charge and discharge current 0.490 mA/cm² over a voltage range of 1.0 to 2.5V. The cycling profile followed a conditioning step of 3 cycles at 0.10 C, followed by 0.19 C (25 cycles), followed by an additional 0.10 C discharge.

### Example: Sulfolane-Based Polymer Electrolyte with an LTO/LFP Coin Cell Battery

A sulfolane-based polymer electrolyte was made comprising the composition outlined in Table 1 below using high-shear mixing in the presence of ceramic media until a translucent light purple polymer solution was obtained. The mixing process was carefully maintained to ensure complete homogeneity but avoid rapid heading and potential material volatilization resulting in compositional deviations.

TABLE 1
Polymer Electrolyte Composition

| | | Chemical | Weight |
|---|---|---|---|
| Chemical | Manufacturer | Description | Percentage |
| Sulfolane | Sigma-Aldrich | Carrier | 76 |
| Bis(trifluoromethane) | Sigma-Aldrich | Lithium salt | 19 |
| sulfonimide lithium salt | | | |
| Poly(ethylene oxide) | MSE Supplies | Polymer | 5 |

The resulting material with the composition outlined within Table 1 was heated to 40° C. to allow for easier transfer to preprepared LFP (lithium iron phosphate) or LTO (lithium iron titanate) electrode discs including the desired active material, multiple carbon sources for short-and long-range conductivity, and a binder for foil adherence. The deposition process of the dry gel polymer electrolyte onto the anode and/or cathode included spreading, though other application methods may also be used as described previously. The electrodes were prepared via a standard doctor blading process where the carbon materials (e.g., Super P and carbon nanotubes) and solvent (NMP) were premixed with the binder (PVDF) to ensure wettability. Subsequently, the active material was added and mixed under high shear rates for 1 h or until complete homogeneity was obtained. The mixing was carefully controlled to ensure complete homogeneity but avoid rapid heating and preferential material volatilization resulting in compositional deviations or solid content from batch to batch. The slurry was coated on the respective foils, copper for the anode and aluminum for the cathode, via a standard doctor blading technique at dried thickness between 60-80 μm depending on the target coating thickness. After drying for 12 hours, the electrode sheets were calendared, and individual electrodes were punched out in preparation for cell assembly. The complete composition of the anode and cathodes can be seen in Table 2.

TABLE 2
Anode (LTO) or Cathode (LFP) Composition

| | | Chemical | Weight |
|---|---|---|---|
| Chemical | Manufacturer | Description | Percentage |
| Lithium Iron | MSE Supplies | Active | 84 |
| Phosphate OR | | Material | |
| Lithium Titanate | | | |
| Super P | MTI | Conductive | 12 |
| | Corporation | Carbon | |
| Poly(vinylidene | OCSiAL | Binder | 4 |
| fluoride) | | | |

After depositing the polymer electrode to both anode and cathode individually, an 18 μm polyolefin separator designed for use in lithium-ion batteries was added in between the two opposing layers of the dry gel polymer electrolyte avoid potential internal short-circuiting. This is not critical; however, it guaranteed that cycling tests was not limited by misalignment during assembly. The cells were crimped closed and conditioned (allowing for the complete polymer electrode wettability of the electrodes) at 37° C. for 12 h prior to cycling. The cycling profile breakdown can be seen in Table 3.

TABLE 3
Polymer Electrolyte Cycling Profile

| Step | Cycle(s) | C-Rate |
|---|---|---|
| 1 | 3 | 0.10 C |
| 2 | 1 | 0.20 C |
| 3 | 1 | 0.30 C |
| 4 | 1 | 0.40 C |
| 5 | 10 | 0.50 C |

| | |
|---|---|
| 6 | Loop Steps 2-6 |

The cells were cycled at 37° C. with a Maccor battery tester (Series 4000) and run until either cycling failure occurred (taken as 80% of first cycle capacity) or 1000 cycles. The results from this example can be seen in Table 4 below, while the cycling results are also plotted in FIG. 7 which represents the specific capacity and Coulombic efficiency per cycle throughout the 1000 cycles at 37° C. of a 2016-coin cell including a LTO anode, LFP cathode, and the proposed sulfolane based dry gel polymer electrolyte.

TABLE 4
2016-Coin Cell Test Results, LTO/LFP with 18 μm
polyolefin separator and dry gel polymer electrolyte.

| | | | | Specific | Specific | |
|---|---|---|---|---|---|---|
| Cycle | | Charge | Discharge | Charge | Discharge | Coulombic |
| Number | | Capacity | Capacity | Capacity | Capacity | Efficiency |
| [#] | C-Rate | [mAh] | [mAh] | [mAh/g] | [mAh/g] | [%] |
| 2 | 0.10 C | 1.51 | 1.45 | 102.71 | 98.82 | 97.40 |
| 152 | 0.20 C | 1.51 | 1.47 | 102.78 | 100.17 | 99.99 |
| 506 | 0.20 C | 1.53 | 1.44 | 103.86 | 99.55 | 100 |
| 1000 | 0.20 C | 1.57 | 1.45 | 106.93 | 98.58 | 100 |

### Example: Sulfolane-Based Polymer Electrolyte with an LTO/LFP Single Layer Pouch Cell Battery

A sulfolane-based polymer electrolyte comprising the composition outlined in Table 1 above was prepared using high-shear mixing in the presence of ceramic media until a translucent light purple polymer solution was obtained. The mixing process was carefully maintained to ensure complete homogeneity but avoid rapid heading and potential material volatilization resulting in compositional deviations. The resulting material with a composition outlined within Table 1 was heated to 40° C. to allow for easier transfer to preprepared LFP (lithium iron phosphate) or LTO (lithium iron titanate) electrode sheets including a mixture of active material, carbon sources, and a binder. The electrodes were prepared via a standard doctor-blading process where the carbon materials and solvent were premixed with the binder to ensure wettability. Subsequently, the active material was added and mixed under high shear rates until complete homogeneity was obtained. Similarly, the mixing during this step was carefully controlled to ensure complete homogeneity but avoid rapid heading and potential material volatilization resulting in compositional deviations or solid content variances. The slurry was coated on the respective foils, copper for the anode and aluminum for the cathode, via a standard doctor blading technique. After drying, the electrode sheets were calendared, and individual electrode were punched out in preparation for cell assembly. The complete composition of the anode and cathodes can be seen in Table 5 below.

TABLE 5
Anode (LTO) or Cathode (LFP) Composition

| | | Chemical | Weight |
|---|---|---|---|
| Chemical | Manufacturer | Description | Percentage |
| Lithium Iron | MSE Supplies | Active | 90 |
| Phosphate OR | | Material | |
| Lithium Titanate | | | |
| Super P | MTI | Conductive | 5.2 |
| | Corporation | Carbon | |
| Carbon nanotubes | TUBALL | Conductive | 0.8 |
| | | Carbon | |
| Poly(vinylidene | OCSiAL | Binder | 4 |
| fluoride) | | | |

After applying a dry gel polymer electrolyte to an exposed surface of each of the anode and cathode, an 18 μm polyolefin separator designed for use in lithium-ion batteries was added in between the two electrode assemblies to avoid internal short-circuiting. While the polyolefin separator is not completely necessary, it helped to guarantee that cycling tests were not limited by erroneous alignment during manual assembly. The cells were sealed within an alumina pouch and conditioned (allowing for the complete polymer electrode wettability of the electrodes) at 37° C. for 12 h prior to beginning cycling. The cycling profile was one that combined two common analytical techniques: a rate capability test and a cycle stability test, into one. The breakdown can be seen in Table 3 above.

The cells were cycled at 37° C. with a Maccor battery tester (Series 4000). The results can be seen in Table 6. FIG. 8 shows the specific capacity and Coulombic efficiency per cycle through 1700 cycles at 37° C. in a 2″×3″ single layer pouch cell including a LTO anode, LFP cathode, and the proposed sulfolane-based dry gel polymer electrolyte.

TABLE 6
2 inch × 3 inch Single Layer Pouch Cell Test Results, LTO/LFP with
18 μm polyolefin separator and dry gel polymer electrolyte.

| | | | | Specific | Specific | |
|---|---|---|---|---|---|---|
| Cycle | | Charge | Discharge | Charge | Discharge | Coulombic |
| Number | | Capacity | Capacity | Capacity | Capacity | Efficiency |
| [#] | C-Rate | [mAh] | [mAh] | [mAh/g] | [mAh/g] | [%] |
| 2 | 0.10 C | 46.18 | 45.77 | 144.19 | 142.92 | 99.12 |
| 100 | 0.50 C | 44.13 | 44.12 | 137.80 | 137.77 | 99.98 |
| 250 | 0.50 C | 44.21 | 44.25 | 138.05 | 138.18 | 100 |
| 500 | 0.30 C | 45.36 | 45.27 | 141.66 | 141.37 | 99.80 |
| 1000 | 0.50 C | 45.13 | 45.12 | 140.93 | 140.89 | 99.97 |
| 1500 | 0.30 C | 44.72 | 44.48 | 139.65 | 138.89 | 99.46 |

### Example: Sulfolane-Based Polymer Electrolyte with an LTO/LFP Double Layer Pouch Cell Battery

A sulfolane-based polymer electrolyte comprising the composition outlined in Table 1 above was prepared using high-shear mixing in the presence of ceramic media until a translucent light purple polymer solution was obtained. The mixing process was carefully maintained to ensure complete homogeneity but avoid rapid heading and potential material volatilization resulting in compositional deviations. The resulting material with a composition outlined within Table 1 was heated to 40° C. to allow for easier transfer to preprepared LFP (lithium iron phosphate) or LTO (lithium iron titanate) electrode sheets including a mixture of active material, carbon sources, and a binder. The electrodes were prepared via a standard doctor-blading process where the carbon materials and solvent were premixed with the binder to ensure wettability. Subsequently, the active material was added and mixed under high shear rates until complete homogeneity was obtained. Similarly, the mixing during this step was carefully controlled to ensure complete homogeneity but avoid rapid heating and potential material volatilization resulting in compositional deviations or solid content variances. The slurry was coated on the respective foils, copper for the anode and aluminum for the cathode, via a standard doctor blading technique. After drying, the electrode sheets were calendared, and individual electrodes were punched out in preparation for cell assembly. The complete composition of the anode and cathodes can be seen in Table 5 above.

After applying a dry gel polymer electrolyte to an exposed surface of each of the anode and cathode stack, an 18 μm polyolefin separator designed for use in lithium-ion batteries was added in between the two stacks of electrode assemblies to avoid internal short-circuiting. While the polyolefin separator is not completely necessary, it helped to guarantee that cycling tests were not limited by erroneous alignment during manual assembly. The cells were sealed within an aluminum pouch and conditioned (allowing for the complete polymer electrode wettability of the electrodes) at 37° C. for 12 h prior to beginning cycling. The cycling profile was one that combined two common analytical techniques: a rate capability test and a cycle stability test, into one. The breakdown can be seen in Table 3 above.

The cells were cycled at 37° C. with a Maccor battery tester (Series 4000). The results can be seen in Table 7. FIG. 9 shows the specific capacity and Coulombic efficiency per cycle through 1500 cycles at 37° C. in a 2 inch×3 inch double layer pouch cell including two LTO anodes, a double-sided LFP cathode, and the proposed sulfolane-based dry gel polymer electrolyte.

TABLE 7
2 inch × 3 inch Double Layer Pouch Cell Test Results, LTO/LFP with
18 μm polyolefin separator and dry gel polymer electrolyte.

| | | | | Specific | Specific | |
|---|---|---|---|---|---|---|
| Cycle | | Charge | Discharge | Charge | Discharge | Coulombic |
| Number | | Capacity | Capacity | Capacity | Capacity | Efficiency |
| [#] | C-Rate | [mAh] | [mAh] | [mAh/g] | [mAh/g] | [%] |
| 2 | 0.10 C | 92.43 | 90.82 | 144.31 | 141.80 | 98.27 |
| 100 | 0.50 C | 91.70 | 91.58 | 143.17 | 142.99 | 99.87 |
| 250 | 0.50 C | 91.59 | 91.57 | 143.01 | 142.97 | 99.97 |
| 500 | 0.40 C | 92.16 | 91.92 | 143.90 | 143.51 | 99.73 |
| 1000 | 0.50 C | 89.63 | 89.61 | 139.95 | 139.91 | 99.97 |

### Example: Sulfolane-Based Polymer Electrolyte with an LTO/LFP Six-Stack Pouch Cell Battery

A sulfolane-based polymer electrolyte comprising the composition outlined in Table 8 was prepared in which the lithium salt, LiN(CF₃SO₂)₂, and the carrier, sulfolane, were mixed using high-shear mixing in the presence of ceramic media until homogeneous and the polymer, PEO, was added incrementally by 1 wt % until a translucent light purple polymer solution was obtained. The mixing process was carefully maintained to ensure complete homogeneity but avoid rapid heading and potential material volatilization resulting in compositional deviations.

TABLE 8
Polymer Electrolyte Composition

| | | Chemical | Weight |
|---|---|---|---|
| Chemical | Manufacturer | Description | Percentage |
| Sulfolane | Sigma-Aldrich | Carrier | 71.25 |
| Bis(trifluoromethane) | Sigma-Aldrich | Lithium salt | 23.75 |
| sulfonimide lithium salt | | | |
| Poly(ethylene oxide) | MSE Supplies | Polymer | 5 |

The resulting material with a composition outlined within Table 8 was heated to 40° C. to allow for easier transfer to preprepared LFP (lithium iron phosphate) or LTO (lithium iron titanate) electrode sheets including a mixture of active material, carbon sources, and a binder. The electrodes were prepared via a standard doctor-blading process where Super P and active material were dry mixed to ensure active material was sufficiently coated with carbon. The carbon nanotubes, binder, and solvent were added subsequently, and mixed under high shear rates until complete homogeneity was obtained. Similarly, the mixing during this step was carefully controlled to ensure complete homogeneity but avoid rapid heading and potential material volatilization resulting in compositional deviations or solid content variances. The slurry was coated on the respective foils, copper for the anode and aluminum for the cathode, via a standard doctor blading technique. After drying, the electrode sheets were calendared, and individual electrodes were punched out in preparation for cell assembly. The complete composition of the anode and cathodes can be seen in Table 5 above.

After spreading one layer of dry gel polymer electrolyte onto an exposed surface of each of the anode and cathode stacks, a 20 μm polyolefin separator designed for use in lithium-ion batteries was added in between the stacks of electrode assemblies to avoid internal short-circuiting. While the polyolefin separator is not completely necessary, it helped to guarantee that cycling tests were not limited by erroneous alignment during manual assembly. The cells were sealed within an aluminum pouch and conditioned at 0.10 C for three cycles to allow for the complete polymer electrode wettability of the electrodes at 37° C. prior to beginning cycling. The breakdown of the cycling profile can be seen in Table 9.

TABLE 9
Polymer Electrolyte Cycling Profile

| Step | Cycle(s) | C-Rate |
|---|---|---|
| 1 | 3 | 0.10 C |
| 2 | 9 | 0.19 C |
| 5 | 10 | 0.19 C |
| 6 | 1 | 0.10 C |

| | |
|---|---|
| 7 | Loop Steps 5-6 |

The cells were cycled at 37° C. with an Arbin battery tester. The results can be seen in Table 10. FIG. 10 shows the specific capacity and Coulombic efficiency per cycle through 160 cycles at 37° C. in a 0.5 inch×1.0 inch six-stack pouch cell including two single-sided and five double-sided LTO anodes, six double-sided LFP cathodes, and the proposed sulfolane-based dry gel polymer electrolyte.

TABLE 10
0.5 inch × 1.0 inch Six-Stack Pouch Cell Test Results, LTO/LFP with
20 μm polyolefin separator and dry gel polymer electrolyte.

| | | | | Specific | Specific | |
|---|---|---|---|---|---|---|
| Cycle | | Charge | Discharge | Charge | Discharge | Coulombic |
| Number | | Capacity | Capacity | Capacity | Capacity | Efficiency |
| [#] | C-Rate | [mAh] | [mAh] | [mAh/g] | [mAh/g] | [%] |
| 2 | 0.19 C | 35.81 | 34.82 | 110.32 | 107.28 | 97.25 |
| 50 | 0.19 C | 34.55 | 34.46 | 106.45 | 106.17 | 99.74 |
| 100 | 0.19 C | 34.86 | 34.88 | 107.38 | 107.46 | 100 |
| 150 | 0.19 C | 35.05 | 35.14 | 107.99 | 108.26 | 100 |

### Example: Sulfolane-Based Polymer Electrolyte with an LTO/LFP Four-Stack Pouch Cell Battery

A sulfolane-based polymer electrolyte comprising the composition outlined in Table 8 above was prepared in which the lithium salt, LiN(CF₃SO₂)₂, and the carrier, sulfolane, were mixed using high-shear mixing in the presence of ceramic media until homogeneous and the polymer, PEO, was added incrementally by 1 wt % until a translucent light purple polymer solution was obtained. The mixing process was carefully maintained to ensure complete homogeneity but avoid rapid heading and potential material volatilization resulting in compositional deviations.

The resulting material with a composition outlined within Table 8 was heated to 40° C. to allow for easier transfer to preprepared LFP (lithium iron phosphate) or LTO (lithium iron titanate) electrode sheets including a mixture of active material, carbon sources, and a binder. The electrodes were prepared via a standard doctor-blading process where Super P, Super C45, and active material were dry mixed to ensure active material was sufficiently coated with carbon. The KS6, carbon nanotubes, binder, and solvent were added subsequently, and mixed under high shear rates until complete homogeneity was obtained. Similarly, the mixing during this step was carefully controlled to ensure complete homogeneity but avoid rapid heading and potential material volatilization resulting in compositional deviations or solid content variances. The slurry was coated on the respective foils, copper for the anode and aluminum for the cathode, via a standard doctor blading technique. After drying, the electrode sheets were calendared, and individual electrodes were punched out in preparation for cell assembly. The complete composition of the anode and cathodes can be seen in Table 11.

TABLE 11
Anode (LTO) or Cathode (LFP) Composition

| | | Chemical | Weight |
|---|---|---|---|
| Chemical | Manufacturer | Description | Percentage |
| Lithium Iron | MSE Supplies | Active | 90 |
| Phosphate OR | | Material | |
| Lithium Titanate | | | |
| Super P | MTI | Conductive | 1.0 |
| | Corporation | Carbon | |
| Super C45 | MSE Supplies | Conductive | 2.1 |
| | | Carbon | |
| KS6 | MSE Supplies | Conductive | 2.1 |
| | | Carbon | |
| Carbon nanotubes | TUBALL | Conductive | 0.8 |
| | | Carbon | |
| Poly(vinylidene | OCSiAL | Binder | 4 |
| fluoride) | | | |

After spreading one layer of dry gel polymer electrolyte onto an exposed surface of each of the anode and cathode stacks, a 20 μm polyolefin separator designed for use in lithium-ion batteries was added in between the stacks of electrode assemblies to avoid internal short-circuiting. While the polyolefin separator is not completely necessary, it helped to guarantee that cycling tests were not limited by erroneous alignment during manual assembly. The cells were sealed within an aluminum pouch and conditioned at 0.10 C for three cycles to allow for the complete polymer electrode wettability of the electrodes at 37° C. prior to beginning cycling. The breakdown of the cycling profile can be seen in Table 12, in which the cell cycled at 0.19 C for 25 cycles followed by an additional 0.10 C discharge.

TABLE 12
Polymer Electrolyte Cycling Profile

| Step | Cycle(s) | C-Rate |
|---|---|---|
| 1 | 25 | 0.19 C |
| 2 | 1 | 0.10 C discharge |

| | |
|---|---|
| 7 | Loop Steps 1-2 |

The cells were cycled at 37° C. with an Arbin battery tester. The results can be seen in Table 13. FIG. 11 shows the specific capacity and Coulombic efficiency per cycle through 96 cycles at 37° C. in a 0.5″×1.0″ four-stack pouch cell including two single-sided and three double-sided LTO anodes, four double-sided LFP cathodes, and the proposed sulfolane-based dry gel polymer electrolyte.

TABLE 13
0.5 inch × 1.0 inch Four-Stack Pouch Cell Test Results, LTO/LFP with
20 μm polyolefin separator and dry gel polymer electrolyte.

| | | | | Specific | Specific | |
|---|---|---|---|---|---|---|
| Cycle | | Charge | Discharge | Charge | Discharge | Coulombic |
| Number | | Capacity | Capacity | Capacity | Capacity | Efficiency |
| [#] | C-Rate | [mAh] | [mAh] | [mAh/g] | [mAh/g] | [%] |
| 2 | 0.19 C | 37.16 | 37.26 | 110.30 | 110.60 | 100 |
| 20 | 0.19 C | 39.75 | 39.71 | 117.99 | 117.88 | 99.91 |
| 40 | 0.19 C | 40.53 | 40.51 | 120.31 | 120.26 | 99.96 |
| 60 | 0.19 C | 40.89 | 40.86 | 121.37 | 121.30 | 99.95 |
| 80 | 0.19 C | 41.02 | 41.00 | 121.76 | 121.70 | 99.95 |

### Example: Nail Penetration

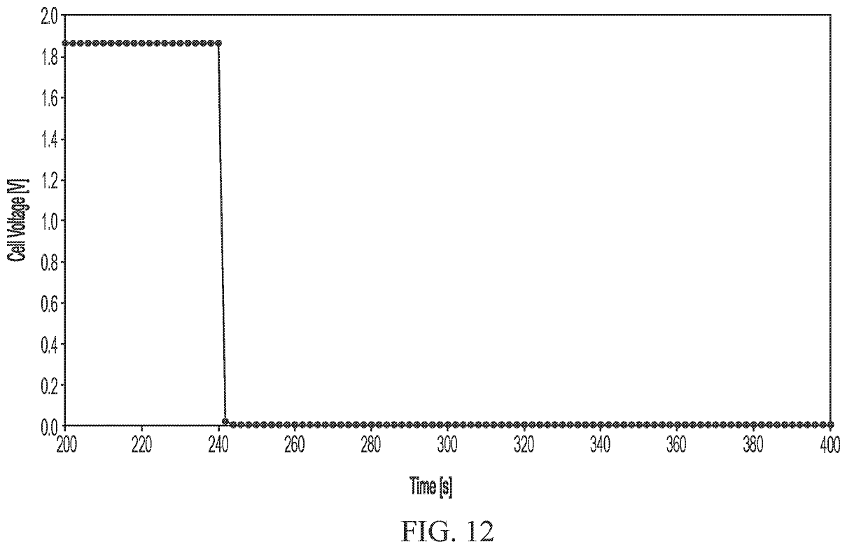

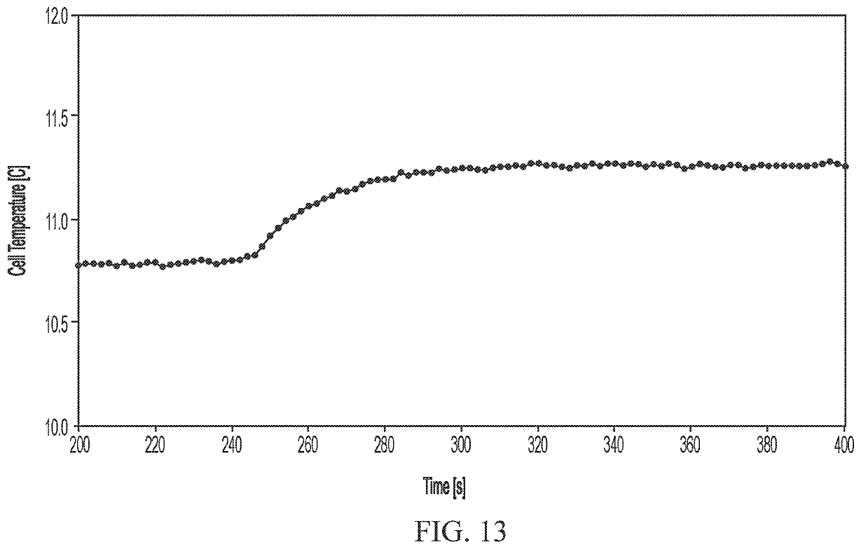

A nail penetration test was performed on a six-stack pouch cell to simulate short-circuiting and assess the safety of the battery. The nail penetration test involved driving a nail, 26 mm in length and 3.06 mm in diameter, through a charged 0.5 inch×1.0 inch six-stack pouch cell including one embodiment of a dry gel polymer electrolyte. FIGS. 12 and 13 show the results of the nail penetration test, cell voltage and cell temperature, respectively. The voltage drops to 0 V and the initial temperature increase of 0.46° C. from 10.80° C. to 11.26° C. results from the short circuit. The gradual temperature increase thereafter results from an increase in ambient temperature. No emissions, fires, or thermal runaway events were observed.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

1. A dry gel polymer electrolyte comprising:
sulfolane;
a high molecular weight polymer mixed with the sulfolane; and
a lithium salt complexed with the high molecular weight polymer.

2. The gel polymer electrolyte of claim 1, wherein the high molecular weight polymer is a high molecular weight polyethylene oxide.

3. The dry gel polymer electrolyte of claim 2, wherein a molecular weight of the high molecular weight polyethylene oxide is between or equal to 1 Mg/mol and 15 Mg/mol.

4. The dry polymer electrolyte of claim 3, wherein a molecular weight of the high molecular weight polyethylene oxide is between or equal to 1 Mg/mol and 5 Mg/mol.

5. The dry gel polymer electrolyte of claim 1, wherein a weight percentage of the high molecular weight polymer relative to an overall weight of the dry gel polymer electrolyte is between or equal to 0.5 wt % and 15 wt %.

6. The dry gel polymer electrolyte of claim 5, wherein the weight percentage of the high molecular weight polymer relative to an overall weight of the dry gel polymer electrolyte is between or equal to 1 wt % and 7.5 wt %.

7. The dry gel polymer electrolyte of claim 1, wherein the high molecular weight polymer is at least one selected from polyethylene oxide, polyacrylonitrile, polyvinylidene fluoride, poly (vinylidene fluoride-co-hexafluoropropylene).

8. The dry gel polymer electrolyte of claim 1, wherein a weight percentage of the sulfolane relative to an overall weight of the dry gel polymer electrolyte is between or equal to 55 wt % and 85 wt %.

9. The dry gel polymer electrolyte of claim 8, wherein the weight percentage of the sulfolane relative to an overall weight of the dry gel polymer electrolyte is between or equal to 65 wt % and 80 wt %.

10. The dry gel polymer electrolyte of claim 1, wherein the lithium salt is at least one selected from LiTFSI, LiPF₆, LiClO₄, LiCF₃SO₃, LiBF₄, LiDFOB, LiTf, and LiAsF₆.

11. The dry gel polymer electrolyte of claim 1, wherein a weight percentage of the lithium salt relative to an overall weight of the dry gel polymer electrolyte is between or equal to 5 wt % and 35 wt %.

12. The dry gel polymer electrolyte of claim 11, wherein the weight percentage of the lithium salt relative to an overall weight of the dry gel polymer electrolyte is between or equal to 15 wt % and 25 wt %.

13. The dry gel polymer electrolyte of claim 1, further comprising a shear media dispersed in the dry gel polymer electrolyte.

14. The dry gel polymer electrolyte of claim 13, wherein the shear media is a at least one selected from a zirconia shear media, an alumina shear media, a ceramic shear media, tungsten carbide shear media, stainless steel shear media, agate shear media, silicon carbide shear media, silicon nitride shear media.

15. An electrochemical cell comprising:
an anode;
a cathode;
at least one dry gel polymer electrolyte layer disposed between the anode and the cathode, wherein the at least one dry gel polymer electrolyte layer comprises a dry gel polymer electrolyte including:
sulfolane;
a high molecular weight polymer mixed with the sulfolane;
a lithium salt complexed with the high molecular weight polymer.

16. The electrochemical cell of claim 15, wherein the high molecular weight polymer is polyethylene oxide.

17. The electrochemical cell of claim 16, wherein a molecular weight of the high molecular weight polyethylene oxide is between or equal to 1 Mg/mol and 15 Mg/mol.

18. The electrochemical cell of claim 17, wherein a molecular weight of the high molecular weight polyethylene oxide is between or equal to 1 Mg/mol and 5 Mg/mol.

19. The electrochemical cell of claim 15, wherein a weight percentage of the high molecular weight polymer relative to an overall weight of the dry gel polymer electrolyte is between or equal to 0.5 wt % and 15 wt %.

20. The electrochemical cell of claim 15, wherein a weight percentage of the high molecular weight polymer relative to an overall weight of the dry gel polymer electrolyte is between or equal to 1 wt % and 7.5 wt %.

21. The electrochemical cell of claim 15, wherein the high molecular weight polymer is at least one selected from polyethylene oxide, polyacrylonitrile, polyvinylidene fluoride, poly (vinylidene fluoride-co-hexafluoropropylene).

22. The electrochemical cell of claim 15, wherein a weight percentage of the sulfolane relative to an overall weight of the dry gel polymer electrolyte is between or equal to 55 wt % and 85 wt %.

23. The electrochemical cell of claim 22, wherein the weight percentage of the sulfolane relative to an overall weight of the dry gel polymer electrolyte is between or equal to 65 wt % and 80 wt %.

24. The electrochemical cell of claim 15, wherein the lithium salt is at least one selected from LiTFSI, LiPF₆, LiClO₄, LiCF₃SO₃, LiBF₄, LiDFOB, LiTf, and LiAsF₆.

25. The electrochemical cell of claim 15, wherein a weight percentage of the lithium salt relative to an overall weight of the dry gel polymer electrolyte is between or equal to 5 wt % and 35 wt %.

26. The dry gel polymer electrolyte of claim 25, wherein the weight percentage of the lithium salt relative to an overall weight of the dry gel polymer electrolyte is between or equal to 15 wt % and 25 wt %.

27. The electrochemical cell of claim 15, further comprising a shear media dispersed in the dry gel polymer electrolyte.

28. The electrochemical cell of claim 27, wherein the shear media is at least one selected from a zirconia shear media, an alumina shear media, a ceramic shear media, tungsten carbide shear media, stainless steel shear media, agate shear media, silicon carbide shear media, silicon nitride shear media.

29. The electrochemical cell of claim 15, wherein the anode comprises at least one selected from lithium titanate, silicon, graphite, lithium, tin, and germanium, and wherein and the cathode comprises at least one selected from lithium iron phosphate, lithium cobalt oxide, lithium nickel manganese cobalt oxide, lithium nickel cobalt aluminum oxide, lithium manganese oxide, and lithium nickel manganese oxide.

30. The electrochemical cell of claim 15, further comprising a separator disposed between the anode and the cathode, wherein the at least one dry gel polymer electrolyte layer comprises a first dry gel polymer electrolyte layer disposed on the anode and a second dry gel polymer electrolyte layer disposed on the cathode with the separator disposed between the first and second dry gel polymer electrolyte layers.

31. The electrochemical cell of claim 15, wherein a thickness of the at least one dry gel polymer electrolyte layer is between or equal to 5 μm and 100 μm.

32. The electrochemical cell of claim 15, wherein the cathode and anode are stacked, wound, or rolled in a parallel or series multi-stack configuration.

33. An implantable medical device comprising the electrochemical cell of claim 15.

34. The implantable medical device of claim 33, wherein the implantable medical device is an implantable portion of a cochlear implant.
